(19) United States
(12) Patent Application Publication
(10) Pub. No.: US 2008/0254469 A1
(43) Pub. Date: Oct. 16, 2008

(54) Method for Regenerating Hydrophilic and Osteophilic Surface of an Implant

(75) Inventor: Takahiro Ogawa, Torrence, CA (US)

Correspondence Address: SQUIRE, SANDERS & DEMPSEY L.L.P., 1 MARITIME PLAZA, SUITE 300, SAN FRANCISCO, CA, 94111, US

(73) Assignee: The Regents of the University of California, Oakland, CA (US)

(21) Appl. No.: 12/067,269

(22) PCT Filed: Aug. 15, 2006
(86) PCT No.: PCT/US06/31964

§ 371 Date: Jun. 30, 2008

Related U.S. Application Data
(60) Provisional application No. 60/719,262, filed on Sep. 20, 2005.

(51) Int. Cl. G01N 33/20; C12Q 1/68; G01N 33/68; B24B 1/00; C23F 1/16; C23F 1/32; C23F 1/02; G01N 21/62; C12Q 1/04

(52) U.S. Cl. 435/6; 435/29; 436/79; 436/80; 436/84; 436/83; 436/86; 436/171; 451/38; 216/100; 216/108

(57) ABSTRACT

Described herein are methods for testing an aged surface on an implant, methods for regenerating a hydrophilic and osteophilic surface on the implant and kits therefor.

### BACKGROUND OF THE INVENTION

1. Field of the Invention

This invention generally relates to method for regenerating hydrophilic and osteophilic metallic implant for biomedical use.

2. Description of the Background

Restoration of skeletal defects or wounds such as femoral neck fracture and spine fusion are common procedures. For example, over 500,000 and 250,000 procedures are performed annually in the U.S. for hip prosthesis implantation and spine fusion surgery, respectively. Meanwhile, about 74 million people in the U.S., which amounts to about 30% of adult population in the U.S., have at least one quadrant of posterior missing tooth that needs to be restored.

Some metallic materials such as titanium are proven biocompatible materials. For example, use of titanium implants has become a standard treatment to replace missing teeth and to fix diseased, fractured or transplanted bone. Restorative treatment of missing teeth using dental implants such as titanium implants have considerable oral health impact, by which masticatory function (Carlsson G E, Lindquist L W, Int. J. Prosthodont 7(5):448-53 (1994); Geertman M E, et al., Community Dent Oral Epidemiol 24(1):79-84 (1996); Pera P, et al., J Oral Rehabil 25(6):462-7 (1998); van Kampen F M, et al., J Dent Res 83(9):708-11 (2004)), Speech (Heydecke G, et al., J Dent Res 83(3):236-40 (2004)) and daily performance and quality of life (Melas F, et al., Int J Oral Maxillofac Implants 16(5):700-12 (2001)) are improved, when compared to the conventional removable denture treatment. In treatments of facial defect resulting from cancer or injury, the use of endosseous implants is crucial to retain the prosthesis (Roumanas E D, et al., Int J Prosthodont 15(4):325-32 (2002)). However, the application of implant therapy in these fields is still limited because of various risk factors including anatomy and quality of host bone (van Steenberghe D, et al., Clin Oral Implants Res 13(6):617-22 (2002)), systemic conditions including diabetes (Nevins M L, Int J Oral Maxillofac Implants 13(5):620-9 (1998); Takeshita F, et al., J Periodontol 69(3):314-20 (1998) and osteoporosis (Ozawa S, et al., Bone 30(1):137-43 (2002)), and ageing (Takeshita F, et al., J Biomed Mater Res 34(1):1-8 (1997)). More importantly, long healing time (about 4-10 months) required for titanium implants to integrate with surrounding bone restricts the application of this beneficial treatment. For example, in the U.S., dental implant therapy has penetrated into only 2% of the potential patients.

In the orthopedic field, the restoration of femoral neck fracture or spine fusion, for example, is a common problem. For example, of over 250,000 procedures performed annually in the U.S. for spine fusion surgery, about 30% or more of patients fail to achieve a solid bony union. The nature and location of bone fracture at these areas do not allow for bone immobilization (e.g., cast splinting) for better healing.

Despite the growing needs of titanium implants, a decent percentage of unsuccessful implants, for instance, ranging 5%-40% in orthopedic implants, and limited application and protracted healing time of implants, particularly in dental implants, are the immediate challenges. Furthermore, implant placements often times have the impaired bone regenerative potential, such as osteoporotic and aged metabolic properties, and thus can lead to difficulty in achieving biological requirements of bone-titanium integration (see, e.g., Ozawa, S. et al. Bone 30, 137-43 (2002); Zhang, H., et al.; J Orthop Res 22, 30-8 (2004); Takeshita, F., et al., J Biomed Mater Res 34, 1-8 (1997); and Yamazaki, M. et al. Oral Surg Oral Med Oral Pathol Oral Radiol Endod 87, 411-8 (1999)).

Freshly prepared titanium surfaces are known to be hydrophilic, while the ones exposed to atmosphere and other liquid for long time become hydrophobic. A hypothetical mechanism is that progressive contamination of carbon and hydrocarbon to the titanium surfaces alters the wettability behavior. Those contaminants can be absorbed onto the titanium surface from the atmosphere and various cleaning solution such as methanol and acetone. However, the osteoconductive potentials, which are crucial for successful implants, in association with the changes in hydrophilicity behavior of implants are unknown and may be reduced with the reduction of hydrophilicity.

Therefore, there is a need for testing the aging of an implant. There is also a need for a method for regenerating hydrophilic as well as osteophilic metallic implant surface.

The embodiments described below address the above identified issues and needs.

### SUMMARY OF THE INVENTION

Provided herein is a method for testing of an aged implant (e.g., metallic implant), which is characterized by surface hydrophobicity and reduced osteoconductive capability (osteophilicity) of the implant and a method for regenerating hydrophilic and osteophilic surface of the implant. Relative to implants with a hydrophobicity and/or osteophobic surface (aged implant), implants with a hydrophilic and/or osteophilic surface have an enhanced tissue integration (osteoconduction) capability.

The method provided herein includes testing the surface hydrophobicity and reduced osteophilicity of an implant and determining the surface as aged if it has a hydrophobic and osteophobic surface. The present invention also provides a method for regenerating a hydrophilic and osteophilic surface on an aged implant that has a hydrophobic and osteophobic surface. The method includes substantially breaking the hydrophobic and osteophobic surface, and removing the hydrophobic and osteophobic surface from the implant or altering the physicochemical properties of the surface.

Kits for testing an aged implant and for regenerating a hydrophilic and osteophobic surface on an implant are also provided herein.

### BRIEF DESCRIPTION OF DRAWINGS

FIGS. 1A-1C show study on the hydrophilicity of the freshly prepared titanium surface and the aged titanium surfaces. In FIG. 1A, the freshly prepared sandblasted surface, 2 week-old sandblasted surface and machined surface were compared. FIGS. 1B and 1C show the test results on their respective time-related attenuating change in hydrophilicity of surfaces prepared by acid-etching and machining.

FIGS. 2A-B show the cell proliferation of osteoblasts on the freshly prepared titanium surfaces and the aged surfaces.

### DETAILED DESCRIPTION

Provided herein is a method for testing of an aged implant (e.g., metallic implant), which is characterized by surface hydrophobicity and reduced osteoconductive capability (osteophilicity) of the implant and a method for regenerating hydrophilic and osteophilic surface of the implant. Relative to implants with a hydrophobicity surface (aged implant), implants with a hydrophilic surface have an enhanced (faster and stronger) tissue integration (osteoconduction) capability.

The method provided herein includes testing the surface hydrophobicity and reduced osteophilicity of an implant and determining the surface as aged if it has a hydrophobic and osteophobic surface. The present invention also provides a method for regenerating a hydrophilic and osteophilic surface on an aged implant that has a hydrophobic and osteophobic surface. The method includes substantially breaking the hydrophobic and osteophobic surface, and removing the hydrophobic and osteophobic surface from the implant or altering the physicochemical properties of the surface.

Kits for testing an aged implant and for regenerating a hydrophilic and osteophobic surface on an implant are also provided herein. The kit can be a stand-alone kit or can be included in a product package that includes the implant.

As used herein, the term “hydrophilicity” refers to an attribute of a material that defines the degree of water affinity of surface of the implant. Hydrophobicity and hydrophilicity are relative terms. Generally, hydrophobicity and hydrophilicity of a surface can be gauged using the Hildebrand solubility parameter δ. The term “Hildebrand solubility parameter” refers to a parameter indicating the cohesive energy density of a substance. The δ parameter is determined as follows:

δ=(ΔE/V)^{1/2}

where δ is the solubility parameter, (cal/cm³)^{1/2};
ΔE is the energy of vaporization, cal/mole; and
V is the molar volume, cm³/mole.

The term “tissue integration capability” refers to the ability of a medical implant to be integrated into the tissue of a biological body through its osteoconductive process. The tissue integration capability of an implant can be generally measured by several factors, one of which is wettability of the implant surface, which reflects the hydrophilicity/oleophilicty (hydrophobicity), or hemophilicity of an implant surface. Hydrophilicity and oleophilicity are relative terms and can be measured by, e.g., water contact angle (Oshida Y, et al., J Mater Science 3:306-312 (1992)), and area of water spread (Gifu-kosen on line text, http://www.gifu-nct.ac.jp/elec/tokoro/fft/contact-angle.html). For purposes of the present invention, the hydrophilicity/oleophilicity can be measured by contact angle or area of water spread on an implant surface described herein relative to the ones of the control implant surfaces. Relative to the implant surfaces not treated with the process described herein, a medical implant treated with the process described herein has a substantially lower contact angle or a substantially higher area of water spread.

### Test of Surface Hydrophilicity and Osteophilicity

In one aspect, the present invention provides a method for testing aging of an implant. The method includes (1) measuring surface hydrophilicity/osteophilicity of an implant and (2) determining the implant as aged if it is hydrophobic and osteophobic.

In some embodiments, the hydrophilicity can be measured by wettability of the surface by water. Wettability can be measured by, for example, contact angle of a water droplet on the surface, discussed above. Where contact angle is measured, a surface can be considered as hydrophilic and hydrophobic if it has a value below about 30 degree and above 30 degree, respectively.

In some embodiments, wettability can be measured using contact angle meter, dynamic hydrophilicity test, and/or various image-analysis-based wettability assessments. Also, measurement of carbon and hydrocarbon content on the surface of implants can be a predictor for the degree of hydrophilicity. For instance, the more the carbon contaminants, the more hydrophobic status progresses. The amount and types of surface elements can be evaluated by X-ray photoelectron spectroscopy (XPS). The amount and types of reactive oxygen species or free radicals that exist on the surface of implants, which can be measured by electron spin resonance spectroscopy for example, may be used to assess the hydrophilicity status. Other means of measuring wettability is to time the period since the manufacture of the implant that gives the estimate of the hydrophilicity status based on the standard reduction curve of hydrophilicity. For instance, the hydrophilicity and the time span after the manufacture of the implant negatively correlate. And thus, the standard regression curve can be created accordingly.

In some embodiments, the osteophilicity of an implant can be measured by various methods and assay systems using culture in vitro examination, including cell response and behavior, particularly of osteoblasts or other osteogenic cells. The assessment can include any of the following: the assessment of the cell attachment, the rate of cell proliferation, the rate of cell differentiation, rate and amount of gene expression, rate of mineralization, protein attachment/adsorption onto the surface or combinations thereof. The osteophilicity can also be measured by various methods and assay systems in the vivo biological body, including the measurement of implant anchorage in tissue, histomorphometric studies of bone formation, X-ray examination of bone formation, or combinations thereof.

In some embodiments, the osteophilicity of an implant can be estimated using a curve of relationship between the age of implants and osteoconductive potential of the implants. For instance, since the osteoconductive potential of implants and time since the implant is prepared negatively correlate, the standard regression curve can be created from the data of several time points, which help estimate the osteoconductive potential of implants with any ages.

In some embodiments, the present invention provides a kit for testing the aging state of an implant. The kit includes a testing agent and a measuring device. The testing agent is preferably water or any bio-inert liquid such as saline solution, alcohol and glycerol, and the measuring device can be any angle measuring device such as image analysis-based methods such as dimensional measurement of the spread area of liquid drop or the assessment of sharpness and clearness of the color or the drawings through the liquid drop, contact angle meter, dynamic hydrophilicity meter. Also, use of chemical indicators that respond to the carbon adsorption to implant surfaces can be the testing devise.

In some embodiments, the kit can be provided along with the implant in a single package or in a separate package.

### Methods for Regenerating Hydrophilic and Osteophilic Surface

In another aspect, the present invention provides a method for regenerating hydrophilic and osteophilic surface of an implant. The method includes providing an implant and treating the surface of the implant with a physical process, chemical process, physicochemical process or combinations thereof. The physical process can be, for example, sand blasting with various particles, such as, but not limited to titanium and aluminum oxide, or machining, turning or filing. The chemical process includes etching the implant with a solution that includes an etching chemical such as an acid or a base and rinsing the implant with water to remove the etching chemical. The physicochemical process includes treating the implant with high energy light that dissolves and removes the surface contaminants, such as carbon and hydrocarbon, that results in the creation of hydrophilic and osteophilic surface.

In some embodiments, the present invention provides a kit for regenerating a hydrophilic and osteophilic surface of an implant. The kit includes a device capable of removing the aged surface layer from or breaking the aged surface layer on an implant so as to regenerate a hydrophilic and osteophilic surface on the implant. In some embodiments, the device is a mechanical device that includes a portion having a rough surface. The rough surface can break the aged surface layer on or remove the aged surface layer from the implant. In one embodiment, the device can be sandblaster or a filing and turning machines.

In some other embodiments, the kit for regenerating a hydrophilic and osteophilic surface of an implant includes an etching agent capable of removing the aged surface layer from or breaking the aged surface layer on an implant so as to regenerate a hydro- and osteophilic surface on the implant. In some embodiment, the etching agent can be an acid or a base. Some exemplary etching agents include, but are not limited to, citric acid, phosphoric acid, sulfuric acid, hydrochloric acid, and nitric acid, fluoric acid and a combination of these.

In some other embodiments, the kit for regenerating a hydrophilic and osteophilic surface includes the high energy light treatment, such as ultra violet light treatment, that removes surface contaminants from the implant surface though its photocatalytic activity.

In some embodiments, the kit can be provided along with the implant or in a separate package.

### Implants

The medical implants described herein include any implants currently available in medicine or to be introduced in the future. The implants can be metallic or non-metallic implants. In some embodiments, the implant can be a metallic implant. In some embodiments, the implant can be a non-metallic implant. Some examples of the non-metallic implant includes, but are not limited to, bone cement implant or a polymer-based implant, such as methymetharcylate-based or polylactic acid-based implants, or bio-glass, ceramic, and zirconium implants. In some embodiments, the non-metallic implants include, for example, calcium phosphate or polymeric implants. Useful polymeric implants can be any biocompatible implants, e.g., bio-degradable polymeric implants that include a polymer or polymer materials. Representative ceramic implants include, e.g., bioglass and silicon dioxide implants. Calcium phosphate implants includes, e.g., hydroxyapatite, tricalciumphosphate (TCP). Exemplary polymers include, e.g., poly-lactic-co-glycolic acid (PLGA), polyacrylate such as polymethacrylates and polyacrylates, poly-lactic acid (PLA) or combinations thereof. In some embodiments, the implant described herein can specifically exclude any of the aforementioned materials.

Useful metallic implants include titanium implants and non-titanium implants. Titanium implants include tooth or bone replacements made of titanium or an alloy that includes titanium. Titanium bone replacements include, e.g., knee joint and hip joint prostheses, femoral neck replacement, spine replacement and repair, neck bone replacement and repair, jaw bone repair, fixation and augmentation, transplanted bone fixation, and other limb prostheses. Non-titanium metallic implants include tooth or bone implants made of gold, platinum, tantalum, niobium, nickel, iron, chromium, cobalt, zirconium, magnesium, magnesium, aluminum, palladium, an alloy formed thereof, e.g., stainless steel, and combinations thereof. Some alloy implants include, but are not limited to, titanium alloy implants, chromium-cobalt alloy implants, platinum alloy implants, nickel alloy implants, stainless steel implants, gold alloy implants, and aluminum alloy implants.

The medical implant described herein can be porous or non-porous implants. Porous implants generally have better tissue integration while non-porous implants have better mechanical strength.

### Medical Use

The medical implants provided herein can be used for treating, preventing, ameliorating, correcting, or reducing the symptoms of a tooth or bone related medical condition by implanting the medical implants in a mammalian subject. The mammalian subject can be a human being or a veterinary animal such as a dog, a cat, a horse, a cow, a bull, or a monkey.

Representative medical conditions that can be treated or prevented using the implants provided herein include, but are not limited to, missing teeth or bone related medical conditions such as femoral neck fracture, missing teeth, a need for orthodontic anchorage or bone related medical conditions such as femoral neck fracture, neck bone fracture, wrist fracture, spine fracture/disorder or spinal disk displacement, fracture or degenerative changes of joints such as knee joint arthritis, bone and other tissue defect or recession caused by a disorder or body condition such as, e.g., cancer, injury, systemic metabolism, infection or aging, and combinations thereof.

The embodiments of the present invention will be illustrated by the following set forth example. All parameters and data are not to be construed to unduly limit the scope of the embodiments of the invention.

### EXAMPLE

### Degrading Osteoconductive Potential of Titanium Over Time and Test for Titanium Aging and Quality Control of Titanium Implants

### Materials and Methods

Titanium sample. Disks (20 mm in diameter and 1.5 mm in thickness) made of commercially pure titanium (Grade 2) were used. The surface of the disks were freshly prepared by machine turning, sandblasting of 50 μm aluminum oxide particles at a distance of 1 cm with a pressure of 3 kg/m, and acid-etching with H₂SO₄.

Hydrophilicity testing. Ten μl of distilled water was gently placed on the titanium surface without physical contact and digitally photographed immediately. The spread area was measured as the area of the water drop in the top view using a digital analyzer (Image Pro Plus, Media Cybernetics, Silver Spring, Md.). The contact angle θ were obtained by the equation: θ=2 tan⁻¹(2h/d), where h and d are the height and diameter of the drop in the side view (Oshida, Y. et al., J Mater Science 3, 306-312 (1992)).

Osteoblastic Cell Culture. Bone marrow cells isolated from the femur of 8-week-old male Sprague-Dawley rats were placed into alpha-modified Eagle's medium supplemented with 15% fetal bovine serum, 50 mg/ml ascorbic acid, 10⁻⁸M dexamethasone, 10 mM Na-β-glycerophosphate and Antibiotic-antimycotic solution containing 10000 units/ml Penicillin G sodium, 10000 mg/ml Streptomycin sulfate and 25 mg/ml Amphotericin B. Cells were incubated in a humidified atmosphere of 95% air, 5% CO₂ at 37° C. At 80% confluency, the cells were detached using 0.25% Trypsin-1 mM EDTA-4Na and seeded onto either the machined titanium or acid-etched titanium disks at a density of 5×10⁴ cells/cm². The culture medium was renewed every three days.

Proliferation assay. To examine the cell proliferation, the osteoblastic cells were incubated on the titanium discs placed on the polystyrene culture dish. The freshly prepared disks and one aged disks of various time periods were used. The cells were gently rinsed twice with PBS and treated with 0.1% collagenase in 300 μl of 0.25% trypsin-1 mM EDTA-4Na for 15 min at 37° C. A hematocytometer was used to count the number of detached cells.

Statistical Analysis. ANOVA was used to examine differences in wettability and cell proliferation variables between the freshly prepared titanium surfaces and aged surfaces; <0.05 was considered statistically significant.

Results

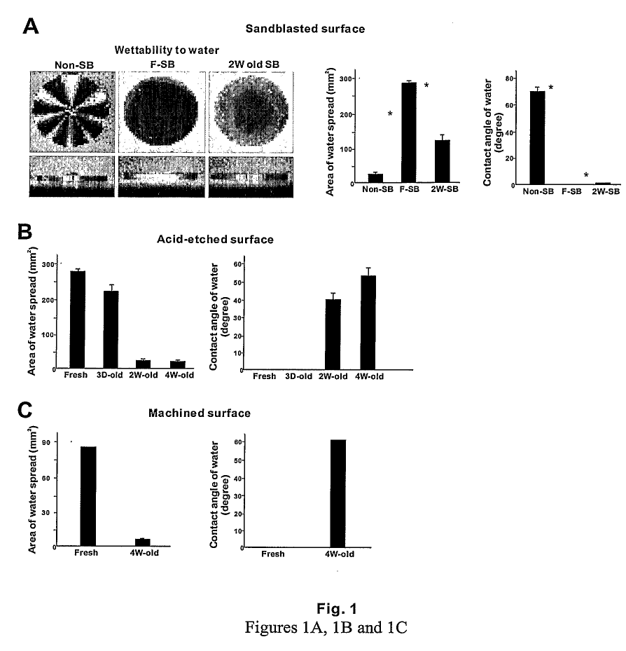

Superhydrophilicity of fresh titanium surface and its reduction with time. Sandblasting on the machined surface changed the wettability behavior from hydrophobic to hydrophilic (FIG. 1A). The spread area of 10 μl water drop increased 13 times after sandblasting of the machined surface. The contact angle of water before sandblasting, which was 69.9°, plummeted to 2.00 after sandblasting, indicating the generation of super-hydrophilic surfaces. The created superhydrophilic property was reduced to 50% level after two weeks in terms of the spread area. The contact angle was increased accordingly with time.

The freshly prepared acid-etched surface and machined surface also showed the superhydrophilic property, which was faded out with time (FIGS. 1B and C). After 4 weeks, the hydrophilic properties at the fresh stage were changed to hydrophobic nature for the both surface types.

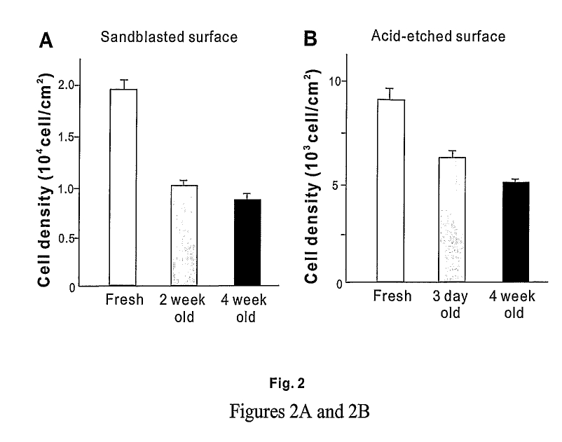

Reducing cell proliferation of osteoblasts on aging titanium. The freshly prepared titanium surfaces showed highest cell proliferation for the different surface types. The cell proliferation was significantly reduced with time for the different surface types (p<0.0001) (FIGS. 2A and 2B). The proliferation was reduced to approximately 50% on the 4 week-old surface compared with the freshly prepared surfaces. While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

What is claimed is:

1. A method for testing surface aging of an implant, comprising
(1) measuring the hydrophobicity and/or osteophobicity of an implant surface, and
(2) designating the surface as aged if the surface is hydrophobic and/or osteophobic.

2. The method of claim 1, wherein an implant surface is hydrophobic and/or osteophobic if it has a contact angle above about 30 degrees.

3. The method of claim 1, wherein the implant is a metallic implant.

4. The method of claim 1, wherein the implant is a titanium implant.

5. The method of claim 3, wherein the metallic implant comprises titanium, gold, platinum, tantalum, niobium, nickel, iron, chromium, cobalt, zirconium, magnesium, magnesium, aluminum, palladium, or an alloy formed thereof.

6. The method of claim 1, wherein the implant is a non-metallic implant.

7. The method of claim 1, wherein the osteophilicity is tested using in vitro or in vivo examination,
wherein the in vitro examination is selected from assessing cell response and behavior of a cell by assessing the cell attachment, the rate of cell proliferation, the rate of cell differentiation, rate and amount of gene expression, rate of mineralization, protein attachment/adsorption onto the surface, or combinations thereof; and
wherein the in vivo examination is selected from measuring implant anchorage in tissue, histomorphometric studies of bone formation, X-ray examination of bone formation, or combinations thereof.

8. The method of claim 7, wherein the cell is an osteoblast or an osteogenic cell.

9. A kit capable of testing the hydrophobicity of an implant, comprising a device for measuring contact angle of the implant.

10. The kit of claim 9, which is in a product package comprising the implant.

11. The kit of claim 9, wherein the implant is a metallic implant or a non-metallic implant.

12. The kit of claim 11, wherein the metallic implant comprises titanium, gold, platinum, tantalum, niobium, nickel, iron, chromium, cobalt, zirconium, magnesium, magnesium, aluminum, palladium, or an alloy formed thereof.

13. A method for regenerating hydrophilicity or osteophilic surface on an implant that has a hydrophobic or non-osteophilic surface, comprising breaking or substantially removing the hydrophobic or non-osteophilic surface.

14. The method of claim 13, wherein the hydrophobic or non-osteophilic surface is substantially broken or removed by a physical process or a chemical process.

15. The method of claim 14, wherein the physical process is sandblasting, machining, or blasting with particles.

16. The method of claim 14, wherein the chemical process is etching with an etching agent.

17. The method of claim 14, further comprising irradiation with ultra violet light.

18. The method of claim 16, wherein the etching agent is an acid or a base.

19. The method of claim 13, wherein the implant is a metallic implant.

20. The method of claim 13, wherein the implant is a titanium implant.

21. The method of claim 19, wherein the metallic implant comprises titanium, gold, platinum, tantalum, niobium, nickel, iron, chromium, cobalt, zirconium, magnesium, magnesium, aluminum, palladium, or an alloy formed thereof.

22. The method of claim 13, wherein the implant is a non-metallic implant.

23. A kit capable of regenerating a hydrophilic surface on an implant, comprising a device or an agent capable of substantially breaking or removing a hydrophobic surface on an implant.

24. The kit of claim 23, wherein the device comprises a rough surface.

25. The kit of claim 24, wherein the device is sandpaper or a file.

26. The kit of claim 23, comprising an etching agent.

27. The kit of claim 26, wherein the etching agent is an acid or a base.

28. The kit of claim 23, which is in a package comprising an implant.

29. The method of claim 23, wherein the implant is a metallic implant or a non-metallic implant.

30. The method of claim 29, wherein the metallic implant comprises titanium, gold, platinum, tantalum, niobium, nickel, iron, chromium, cobalt, zirconium, magnesium, magnesium, aluminum, palladium, or an alloy formed thereof.
